(19) United States
(12) Patent Application Publication
(10) Pub. No.: US 2024/0096503 A1
(43) Pub. Date: Mar. 21, 2024

(54) SYSTEMS AND METHODS FOR QUALITY INSPECTION AND REGULATION ADHERENCE

(71) Applicant: Binh T. Nguyen, Westminster, CA (US)

(72) Inventor: Binh T. Nguyen, Westminster, CA (US)

(21) Appl. No.: 18/518,310

(22) Filed: Nov. 22, 2023

Related U.S. Application Data
(60) Provisional application No. 63/496,022, filed on Apr. 14, 2023.

(51) Int. Cl. G16H 70/20; G06Q 50/26

(52) U.S. Cl. CPC G16H 70/20; G06Q 50/265

(57) ABSTRACT

Provided herein are systems and methods for collaboratively assessing adherence to pharmaceutical regulations and performing inspections of a pharmaceutical facility, operable on a system including a computing device having a database server, a processor, a display, and a camera, wherein the systems and methods include performing data pulls by pulling information from a plurality of sources, pulling regulatory guidelines from a plurality of official regulatory bodies, and checking for updates to the information and the regulatory guidelines at predetermined intervals; such that the data received from the data pulls is used to assess the adherence to pharmaceutical regulations, perform the inspections, and answer input queries from a user related to the information and the regulatory guidelines.

### CROSS-REFERENCE TO RELATED APPLICATIONS

This application claims the benefit of U.S. Provisional Application No. 63/496,022, filed Apr. 14, 2023, which is hereby incorporated by reference, to the extent that it is not conflicting with the present application.

### BACKGROUND OF INVENTION

### 1. Field of the Invention

The invention relates generally to regulatory quality assurance and inspection, and more particularly to quality inspection and adherence to regulations and protocols of various worldwide regulatory bodies covering commodities such as pharmaceuticals, medical devices, biologics, foods, and tobacco products.

### 2. Description of the Related Art

Many regulators exist worldwide for the purpose of regulating products such as pharmaceuticals, biologics, medical devices, dietary supplements, food, and so on. Such regulatory bodies include the United States Food and Drug Administration (USFDA), The Ministry of Food and Drug Safety (MFDS), Health Canada, European Medicines Agency (EMA), for example. It can be challenging and complex to understand the regulations set out by each individual regulatory body, particularly when an individual, company, or any entity is setting out to market products into different countries. There is a lot of data generated throughout different regulatory bodies, provided as public information, including subscribed data and scientific data. It can be difficult to obtain and collect such information in order to gain intelligence regarding producing high-quality, regulated products. Additionally, it can be challenging or time consuming to have on-site inspections performed for quality checks ensuring that regulations are being followed. Travel restrictions can also hinder the ability to perform on-site inspections. Therefore, there is a need for a solution to the above problems.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

Provided herein are systems and methods for inspection and regulation adherence of biologics and pharmaceuticals, wherein the inspection can be performed remotely or on-site using a paperless, electronic system. This software may be referred to as a software for a system for assessing adherence to pharmaceutical regulations and performing inspections of pharmaceutical facilities, for example. This software and associated system may be referred to as “eQip” or “ieQip” generally referring to “electronic quality inspection program/protocol” for example. In some embodiments, software is provided for assisting in inspections. For illustration purpose, pharmaceutical commodity is to be used as a model for other commodities to follow. Generally, as used herein, “pharmaceutical” is used as a term relating to medicinal drugs, or their preparation, manufacture, use, or sale.

In some embodiments, a system is provided comprising a software for product regulation and inspection, wherein the software compiles regulations from all over the world into a single platform such that companies, individuals, and other entities can better and more easily understand the regulations and be prepared to market their products globally. In such embodiments, the regulations are related to pharmaceuticals, drugs, biologics, medical devices, dietary supplements, foods, and so on.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 depicts an example of a user interface that may be shown to a user while using the software for electronic quality inspection, showing various countries' platforms that are accessible using the software, according to an aspect.

FIGS. 2A-2B depict two examples of a user interface that may be shown to a user while using the software for electronic quality inspection, showing information gathering via the artificial tool provided in the software, according to an aspect.

FIG. 3 depicts an example of a user interface that may be shown to a user while using the software for electronic quality inspection, showing a dashboard 108, according to an aspect.

FIG. 4 depicts an example of a user interface that may be shown to a user while using the software for electronic quality inspection, showing a firms page, according to an aspect.

FIG. 5 depicts an example of a user interface that may be shown to a user while using the software for electronic quality inspection, showing a detailed page of an assignment, according to an aspect.

FIG. 6 depicts an example of a user interface that may be shown to a user while using the software for electronic quality inspection, showing an inspection report, according to an aspect.

FIG. 7 is a diagram showing a high level overview of the system architecture of the software for electronic quality inspection, according to an aspect.

FIG. 8 is a diagram showing the general structure of the Postgres database of the software for electronic quality inspection, according to an aspect.

FIG. 9 is a flow chart showing an exemplary process for logging into the software for electronic quality inspection, according to an aspect.

FIG. 10 is a flow chart showing an exemplary process for a user accessing the software for electronic quality inspection as an individual, according to an aspect.

FIG. 11 is a flow chart showing an exemplary process for a user accessing the software for electronic quality inspection on behalf of or acting as a firm, according to an aspect.

FIG. 12 is a flow chart showing an exemplary process for creation of an assignment by using the software for electronic quality inspection, according to an aspect.

FIG. 13 is a flow chart showing an exemplary process for performing an inspection by using the software for electronic quality inspection, according to an aspect.

FIG. 14 is a flow chart showing an exemplary process for performing a lookup using the software for electronic quality inspection, according to an aspect.

FIG. 15 is a flow chart showing an exemplary process for adding information to a database of the software for electronic quality inspection, according to an aspect.

FIG. 16 is a flow chart showing an exemplary process for asking questions and receiving answers from the AI database of the software for electronic quality inspection, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

It should be understood that, for clarity of the drawings and of the specification, some or all details about some structural components or steps that are known in the art are not shown or described if they are not necessary for the invention to be understood by one of ordinary skills in the art.

As used herein and throughout this disclosure, the term “mobile device” refers to any electronic device capable of communicating across a mobile network. A mobile device may have a processor, a memory, a transceiver, an input, and an output. Examples of such devices include cellular telephones, personal digital assistants (PDAs), portable computers, etc. The memory stores applications, software, or logic. Examples of processors are computer processors (processing units), microprocessors, digital signal processors, controllers and microcontrollers, etc. Examples of device memories that may comprise logic include RAM (random access memory), flash memories, ROMS (read-only memories), EPROMS (erasable programmable read-only memories), and EEPROMS (electrically erasable programmable read-only memories). A transceiver includes but is not limited to cellular, GPRS, Bluetooth, and Wi-Fi transceivers.

“Logic” as used herein and throughout this disclosure, refers to any information having the form of instruction signals and/or data that may be applied to direct the operation of a processor. Logic may be formed from signals stored in a device memory. Software is one example of such logic. Logic may also be comprised by digital and/or analog hardware circuits, for example, hardware circuits comprising logical AND, OR, XOR, NAND, NOR, and other logical operations. Logic may be formed from combinations of software and hardware. On a network, logic may be programmed on a server, or a complex of servers. A particular logic unit is not limited to a single logical location on the network.

Mobile devices communicate with each other and with other elements via a network, for instance, a cellular network. A “network” can include broadband wide-area networks, local-area networks, and personal area networks. Communication across a network can be packet-based or use radio and frequency/amplitude modulations using appropriate analog-digital-analog converters and other elements. Examples of radio networks include GSM, CDMA, Wi-Fi and BLUETOOTH.RTM. networks, with communication being enabled by transceivers. A network typically includes a plurality of elements such as servers that host logic for performing tasks on the network. Servers may be placed at several logical points on the network. Servers may further be in communication with databases and can enable communication devices to access the contents of a database. For instance, an authentication server hosts or is in communication with a database having authentication information for users of a mobile network. A “user account” may include several attributes for a particular user, including a unique identifier of the mobile device(s) owned by the user, relationships with other users, call data records, bank account information, etc. A billing server may host a user account for the user to which value is added or removed based on the user's usage of services. One of these services includes mobile payment. In exemplary mobile payment systems, a user account hosted at a billing server is debited or credited based upon transactions performed by a user using their mobile device as a payment method.

For the following description, it can be assumed that most correspondingly labeled elements across the figures (e.g., 105 and 205, etc.) possess the same characteristics and are subject to the same structure and function. If there is a difference between correspondingly labeled elements that is not pointed out, and this difference results in a non-corresponding structure or function of an element for a particular embodiment, example or aspect, then the conflicting description given for that particular embodiment, example or aspect shall govern.

Provided herein are systems and methods for quality inspection and regulation adherence, particularly regulations relating to the development, manufacturing, and sale of pharmaceuticals, biologics, medical devices, tobacco, dietary supplements, food, and so on, comprising a software for electronic quality inspection. In some embodiments, the systems provided herein comprise software for collecting and providing data related to such regulations. In some embodiments, a platform is provided, wherein the platform is used for accessing the software. In some embodiments, the software provides regulations from different regulatory bodies across the world. In some embodiments, the software is accessible by auditors for conducting inspections. In some embodiments, the auditors are third party inspectors or consultants. In some embodiments, the auditors are internal auditors of a company. In some embodiments, the software provided herein is efficient and effective in collecting data in real time for the purpose of remote inspection and assessment.

In some embodiments, a system is provided comprising a software related to product regulation, wherein the software incorporates artificial intelligence tools to pull data globally to help companies make critical decisions in research, development, and manufacturing. In some embodiments, the software can help alleviate problems such as drug shortages, manufacturing deficiencies, and help to advance scientific progress. In some embodiments, data relating to regulations and science can be easily retrieved and understood by users. In some embodiments, the software provides the user with a guided experience, such that a user can more easily and efficiently perform the inspection process. Additionally, the guided software can help to reduce inspection time and report writing time, and can be accessed through a tablet, laptop, or any suitable mobile device. In some embodiments, the software can allow for multiple auditors, such as multiple auditors having different types or levels of expertise, to collaborate simultaneously on a particular project, which can also help to make the inspection more efficient.

In some embodiments, the software provided herein can help companies stay on top of their manufacturing processes and controls. From a global perspective, this can help to ensure that quality drugs are continuously available on the market and accessible to patients worldwide.

In some embodiments, the software provided herein can allow multiple users to enter data at the same time while on-site or while remotely conducting audits. In some embodiments, managers and owners can log in remotely to the software in order to track or monitor progress in real time.

In some embodiments, the software provided herein can act as a training platform for regulators and companies, such that various entities can cooperate to improve patient care worldwide. For example, the software provided herein can guide learners through the process of actually conducting an inspection, how to apply regulations, and how to develop necessary evidence to build a case.

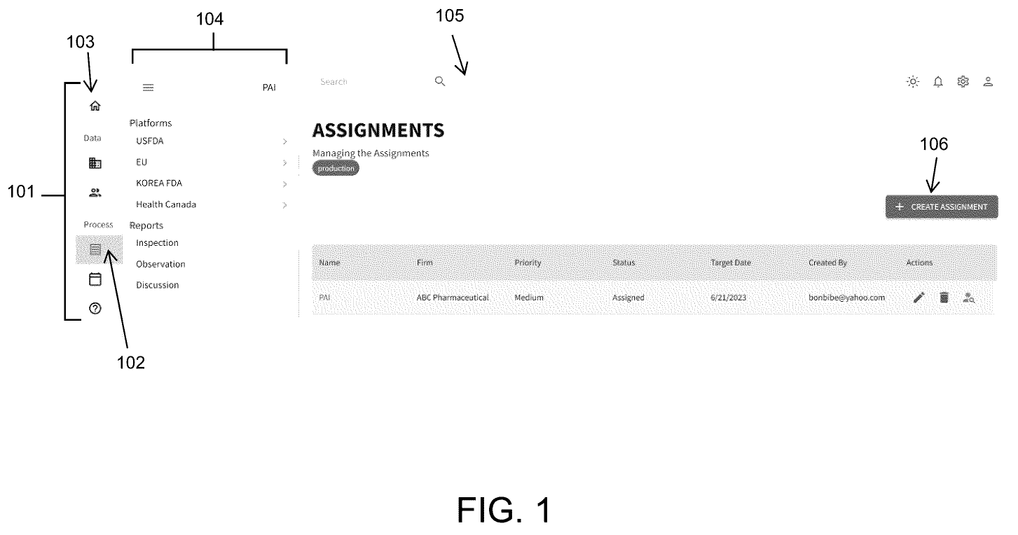

FIG. 1 depicts an example of a user interface that may be shown to a user while using the software for electronic quality inspection, showing various countries' platforms that are accessible using the software, according to an aspect. A user interface may show a main menu 101, comprising a plurality of menu items for navigation, such as, for example, a dashboard menu item 103, which can bring the user to a home or dashboard screen, and an assignments menu item 102. Certain menu items can open a sub-menu for that particular menu item, such as the example shown. The assignments menu item 102 is shown as being selected and is therefore highlighted, and the assignments sub-menu 104 is shown as being displayed to the user. Regulations for various regulatory bodies, such as the USFDA, EMA, MFDS, and Health Canada can then be accessed through the process sub-menu 104. It should be understood that regulations from any regulatory body can also be added to or accessed through the software provided herein.

The user interface may also display a main panel 105 in which various pages can be displayed according to the menu item selected. As an example, the main panel 105 is displaying a list of assignments in FIG. 1. Assignments can be created and modified by authorized users. The assignment main panel may also include a button for creating a new assignment 106. Assignments can also be referred to as work orders, projects, or any other suitable term. In such embodiments, an assignment is created to be specific to a particular company. One assignment may request multiple audits, such as, for example, audits for both USFDA and Health Canada. In such cases, regulations from both countries would be turned on. It should be understood that any number of regulations could be requested and accessed for any one or more assignments. It should also be understood that certain entities may use more than one assignment for the review, assessment, or audit of any particular practice, pharmaceutical, facility, and so on.

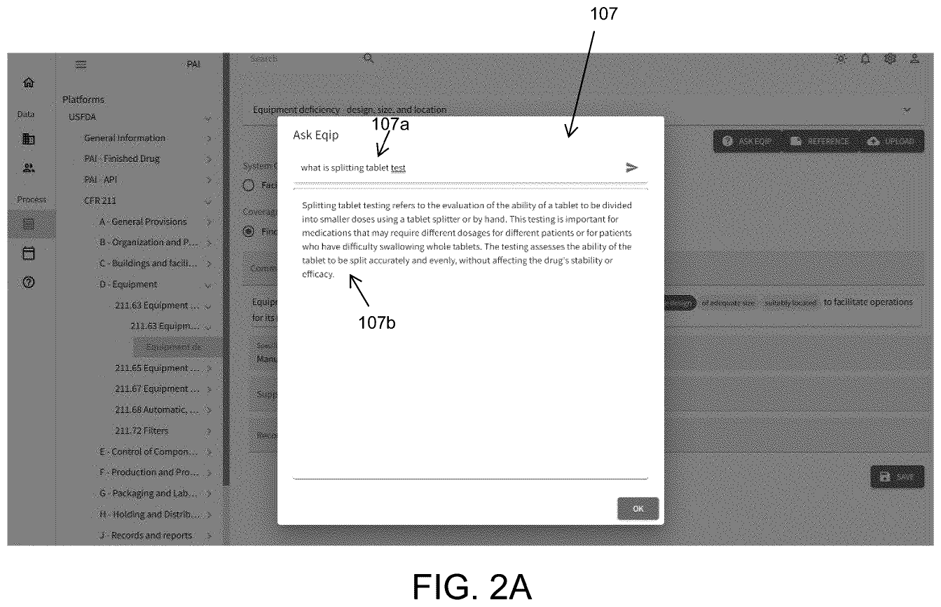

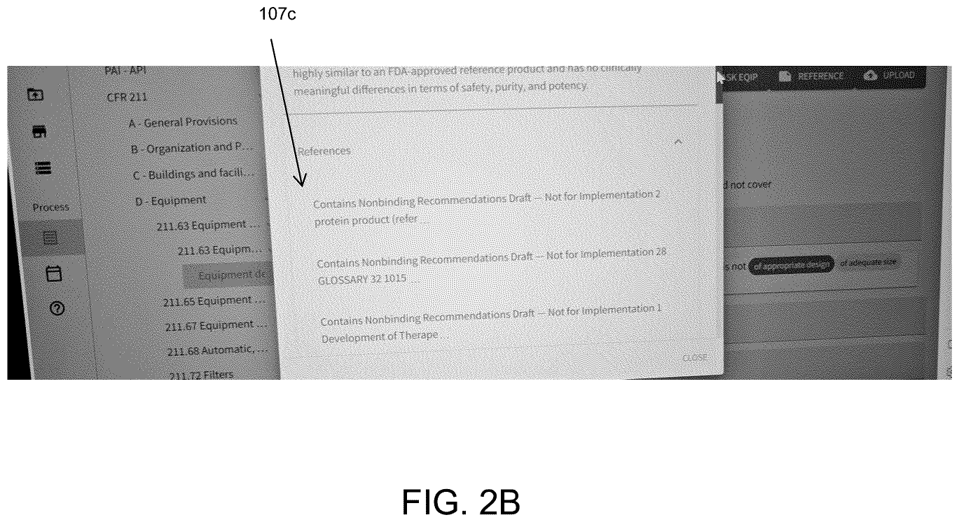

FIGS. 2A-2B depict two examples of a user interface that may be shown to a user while using the software for electronic quality inspection, showing information gathering via the artificial tool provided in the software, according to an aspect. In some embodiments, artificial intelligence provided in the software can run data loaded from various references in order to quickly provide answers to users. For example, a user can input a question, and receive a response as shown in the question pop-up box 107. An exemplary question relating to a “splitting tablet test” is shown by 107a. A response provided by the software is shown by 107b.

In some embodiments, the software can also provide available resources 107c to the user such that the user can check for accuracy, gather additional data, or conduct their own research. For example, the artificial intelligence tools of the software can provide answers to users in response to user questions, and next provide references to the user. The user can then click through any of the provided references for more information on the requested topic or question.

Generally, the above can be an extremely beneficial tool to companies to quickly gather intelligence from a wide variety of references and data sources. This can also allow users to learn from previous mistakes made by other users or companies. For example, some companies may be cited by different regulators around the world when not adhering to certain guidelines, which can serve as a helpful tool for users of the software. The data sourced by this software can also be used by regulators to compare regulations between different bodies, and learn where individual countries stand with respect to each other in regulating the quality of certain products. Also, data about inspectors/auditors/investigators from various regulatory bodies can be gathered to learn about them and their inspection styles, and capabilities, and be provided to the entity in need of an inspection before they show up for inspections.

In some embodiments, data can be loaded into the software for quick reference, data pull, and answers, such as the answer shown by 107b. As an example, data may be pulled daily and uploaded into the software. It should be understood that data pulls could be performed at any suitable interval and the software can be updated at any suitable interval for users. For example, updates to the software could be pushed to users on a regular basis, or on a more frequent basis and then change to less frequent updates as needed and based on availability of new data. As an example, the software update pushes to the user could be first on a monthly basis, then quarterly, then annually. Again, it should be understood that these updates may be based on availability of new and accurate data. Such updates could be from public sources, subscription sources, requested sources, and so on. This data can then be used for providing answers to users, as discussed above, and for providing guidelines and regulations to auditors. In some embodiments, subscribed data can be included in the software for providing information to users. Subscribed data may include, as an example, information from sources that is purchased, licensed, or the like. For example, United States Pharmacopeia (USP) may be embedded into the software. In some embodiments, purchase of the software for product regulation and inspection may include access to such data which would ordinarily be accessible to users through purchase or subscription.

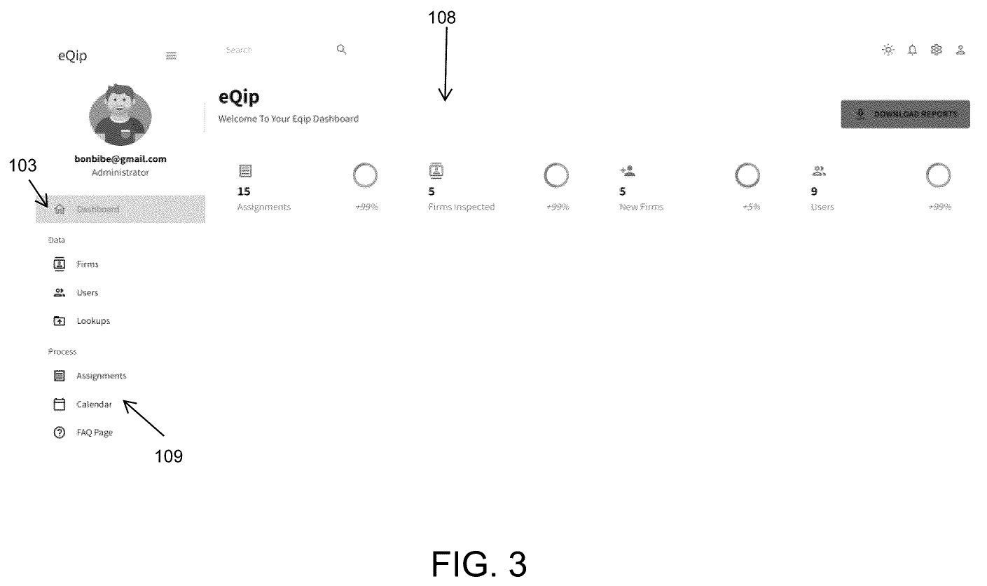

FIG. 3 depicts an example of a user interface that may be shown to a user while using the software for electronic quality inspection, showing a dashboard 108, according to an aspect. The dashboard 108 may act as a main page for inspection, for example. When the dashboard menu item 103 is selected and highlighted, the dashboard 108 may be shown in the main panel. As an example, the dashboard 108 may display assignments and number of total assignments, firms and total number of firms, total number of new firms, and users associated with the assignments. In some embodiments, a calendar 109 is accessible by a logged in user.

In some embodiments, the users may be provided with various roles, such as, for example, administrator, manager, supervisor, owner, lead auditor, assisting auditor, and so on. Depending on the role assigned to a user, the user can have their own access privileges allowing access to various aspects of the software or information related to the assignment or company. Privileges may be created or customized under a parent company, a subsidiary of a parent company, or just for a particular assignment within a parent company, or subsidiary of a parent company.

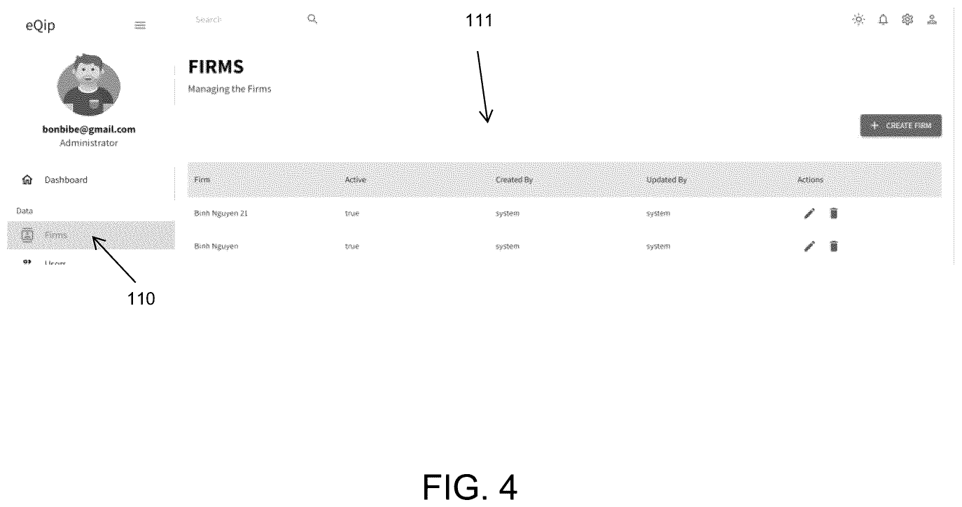

FIG. 4 depicts an example of a user interface that may be shown to a user while using the software for electronic quality inspection, showing a firms page 111, according to an aspect. Selection of the firms menu item 110 may display a firms page 111 in the main panel as shown as an example. The firms page 111 may display a list of firms to be inspected or audited, or in the process of being inspected or audited.

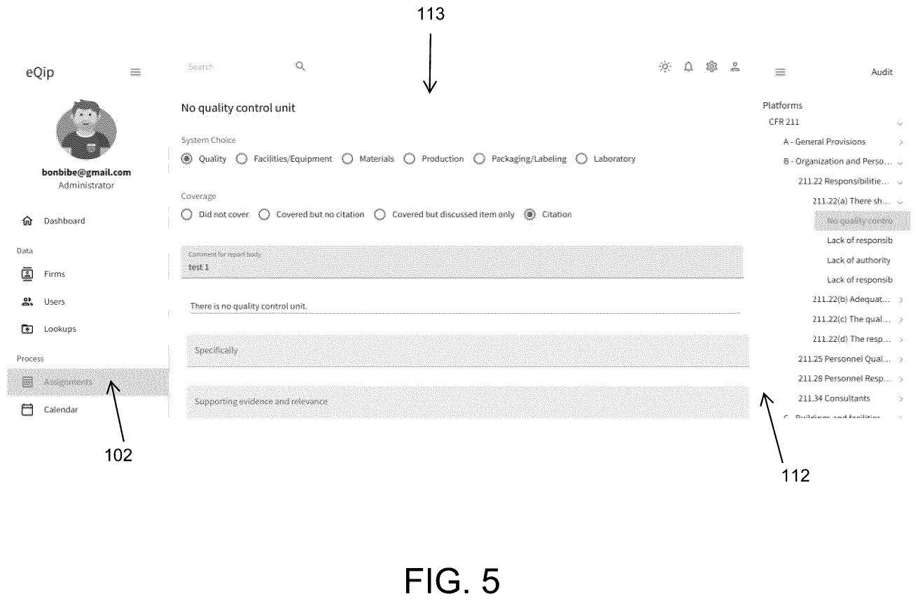

FIG. 5 depicts an example of a user interface that may be shown to a user while using the software for electronic quality inspection, showing a detailed page of an assignment, according to an aspect. The assignments menu item 102 is selected, and a user can then select a single assignment to input details and review regulatory requirements. Regulatory requirements can be viewed in a side panel 112, for example. Within a single selected assignment 113, details relating to the inspection or audit can be entered.

In some embodiments, such user interfaces may be accessed and used during remote inspection, for example. In some remote inspections, a first user may be present at the site to be inspection, and a second user, who may be an auditor, may be remote with respect to the site and may guide the first user during the inspection. The auditor may guide the first user to walk through the facility using a camera. In such embodiments, the camera may be associated with the device providing the software for inspection, or may be a separate camera. Interviews may then be conducted in real time, and answers can be recorded int the software along with upload of relevant evidence, documents, photos, and so on.

In some embodiments, data can be pre-defined with in compliance responses such that once the auditor determines that a particular regulation is not violated, the auditor can check a box “yes” and software will populate a generic answer, allowing the system to type pre-defined answers, which can save time for the auditor while maintaining consistency.

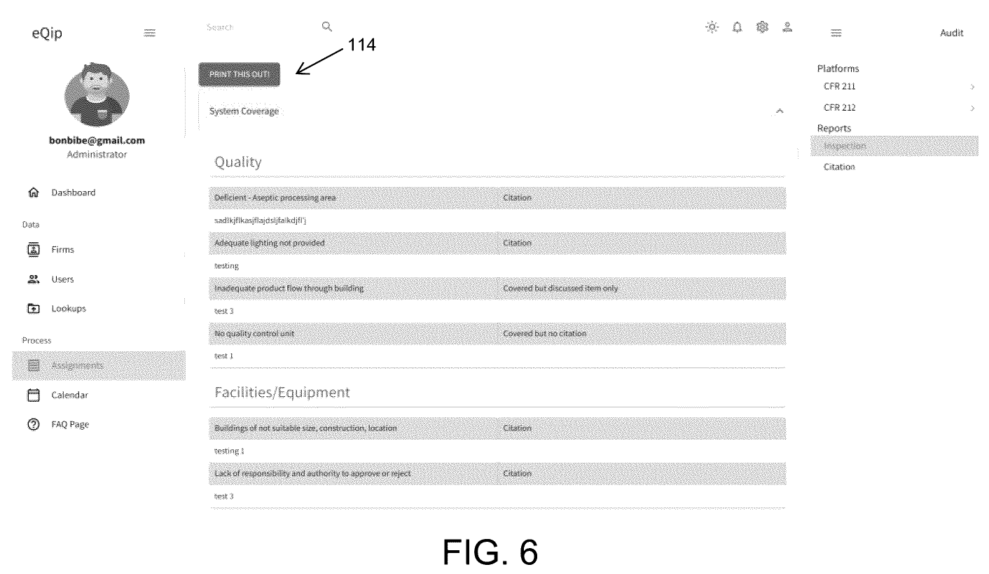

FIG. 6 depicts an example of a user interface that may be shown to a user while using the software for electronic quality inspection, showing an inspection report, according to an aspect. After completion of an assignment, a summary of the inspection or audit may be generated in the form of an inspection report. This can then be printed using a print button 114 or can be electronically shared with other users.

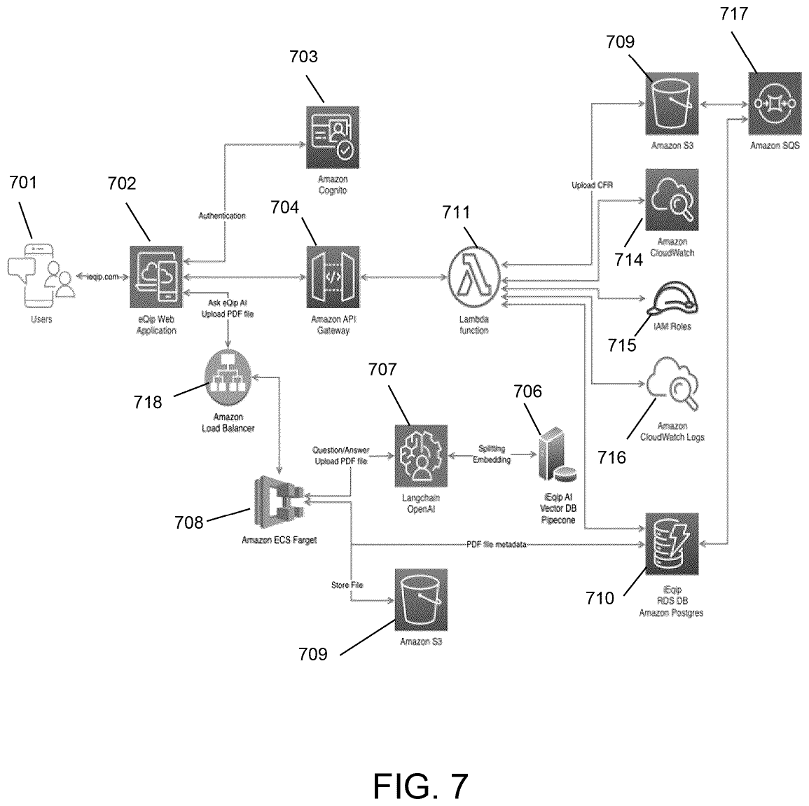

FIG. 7 is a diagram showing a high level overview of the system architecture of the software for electronic quality inspection, according to an aspect. Generally, it should be understood that arrows depicted as interconnecting various systems, databases, users, and so on, are indicated as being in data communication with each other. For example, double-ended arrows may indicate that data can be pushed and pulled between the two elements being connected by the double-ended arrows.

Users 701 may access the software for electronic quality inspection, also referred to herein as “eQip,” by accessing an eQip web application 702 via any suitable web-enabled device. Authentication of a user may be performed by Amazon Cognito 703 or any suitable authenticator which stores information relating to login of users, authenticating of users, and so on. The eQip web application 702 may be in communication with the Amazon API gateway 704, with which “Ask eQip AI” functionality can be performed. Other system components relating to users accessing the system may include the Amazon Load Balancer 718 which may serve to manage users such that the system is able to scale users and servers on which the users are hosted, such that the system can manage, scale, and balance users for speeding up of services of the system by dividing up user traffic between various servers, for example.

The system may allow for direct upload of information, such as, for example, data which can include guidelines by regulatory bodies and information on pharmaceuticals. A user 701 accessing the system through a web application 702 can directly upload a file, such as a PDF, which is stored in a file storage system such as the Amazon S3 system component 709. The Amazon S3 709 can also store files such as spreadsheets (for example, in csv format) which allow for the generation of templates and pages for users to fill in inspection reports, and the like. Once a user has uploaded an information file into the Amazon S3 709, the uploaded information is then stored in at least two places: first, the original file is stored in the Amazon S3 file storage system 709, and second, the system may be configured to automatically process the information in the uploaded file such that the file undergoes a deep learning process, and the text contained within the file can be converted into binary and stored in the vector-based database 706. The vector-based database 706 may be a Pinecone database, for example. The vector-based database 706 may be in communication with a Longchain OpenAI system component 707, a system component which allows a generative AI system such as, but not limited to, ChatGPT, to be in communication with and use data from the system's vector-based database 706 to generate answers to questions inputted by the user. The Longchain OpenAI system component 707 may be in communication with the Amazon ECS Farget 708 system component, which is also referred to as the AWS serverless container or cloud server to host the software.

The Amazon ECS Farget 708 system component may also be in communication with the Amazon S3 709 system component, and the Amazon RDS Postgres database 710 system component. The database 710 may retain all data input in the system and may be used for storage of all information relating to transactions in the system. “Transactions” may, for example, refer to information relating to filled forms (such as filled inspection reports), and any other information relating to inspection or audit activity filled into templates generated by the spreadsheets hosted in file storage system 709. That is, the system may be configured to create fillable forms, templates, and so on based on the spreadsheets stored in the Amazon S3 709. A user 701 can be provided with a fillable form, template, guide, or the like in order to perform an inspection or audit specific to a regional regulatory guideline or CFR, for example. The data inputted by the user 701 into the form, template, or guide can then be stored in database 710. However, it should be understood that, generally, files uploaded by the user may be stored in the file storage system 709. For example, a user may take photos and upload photos relating to an inspection or assignment into the system. Such files can then be stored in a particular bucket of the Amazon S3 709.

The Amazon API gateway 704 system component may be in communication with the Lambda function 711 system component. The Lambda function 711 system component may host several services, such as, for example: assignment, firm, users/user management, inspection, and so on. This component may also be in communication with the following system components: the Amazon S3 709 system component, the Amazon RDS Postgres database 710 system component, the Amazon CloudWatch 714 system component, which serves to assist with login trouble shooting (i.e., system errors) by the developer, the IAM Roles 715 system component, which serves to access all AWS services (database, Lambda, Postgres, Farget, and so on), and the Amazon Cloudwatch Logs 716 system component, which serves to log the Amazon Cloudwatch and create and store system logs for the developer for the purposes of troubleshooting, for example. The Amazon SQS system component 717 may be in communication with the Amazon S3 709 and the Postgres database 710 and may serve to manage a queue system. That is, the Amazon SQS 717 can create and manage queues such that system functionality can be scaled and distributed according to the system's needs, for example.

Generally, the various technologies used for the software for electronic quality inspection may include, but is not limited to, the following technologies. The front end may use JavaScript and ReactJS. The back end may use JavaScript, NodeJS, ExpressJS, AWS API Gateway, AWS Lambda. The database may use Postgres. The AI may use OpenAI, and ChatGPT models. The vector database may use Pinecone, or any other suitable vector-based database having the ability to perform search and similarity matching. The cloud provider may be AWS. The source control may be GitHub. The build pipeline may be GitHub Actions. The source code may be adapted from GitHub to provide the source code of the eQip system, for example.

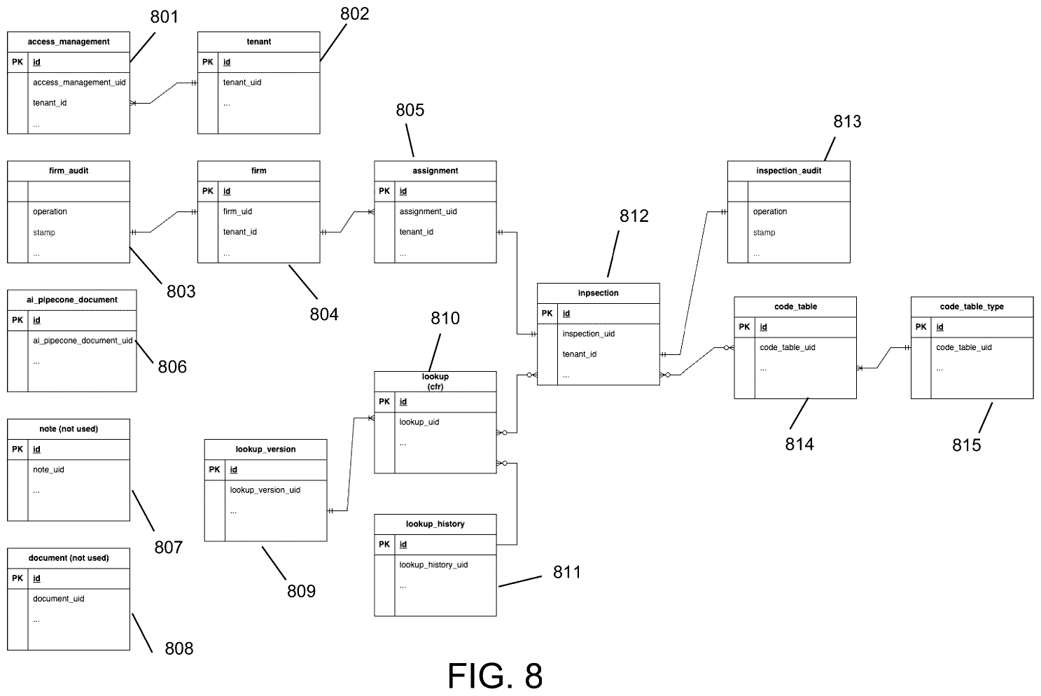

FIG. 8 is a diagram showing the general structure of the Postgres database (shown as 710 in FIG. 7) of the software for electronic quality inspection, according to an aspect. Generally, this database may comprise sections for access management 801, tenants 802, firm audits 803, firms 804, assignments 805, AI Pinecone documents 806, notes 807, documents 808, lookup version 809, lookup 810, lookup history 811, inspections 812, inspection audits 813, code tables 814, and code table types 815. The diagram illustrates the storage of data in the system and each section of data in relation to the others, such as, for example, assignments 805 being associated with firms 804, as well as with inspections 812, and so on.

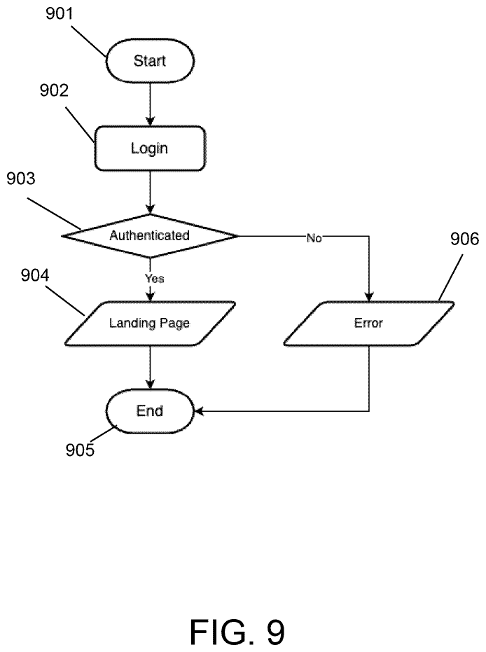

FIG. 9 is a flow chart showing an exemplary process for logging into the software for electronic quality inspection, according to an aspect. To start the process (step 901), the user may login to the eQip web application (step 902), and become authenticated as an authorized user (step 903). If the authentication process confirms that the user is an authorized user, the user is directed to a landing page (step 904) and can complete the process of logging in (step 905). If the authentication is not successful, an error is generated (step 906).

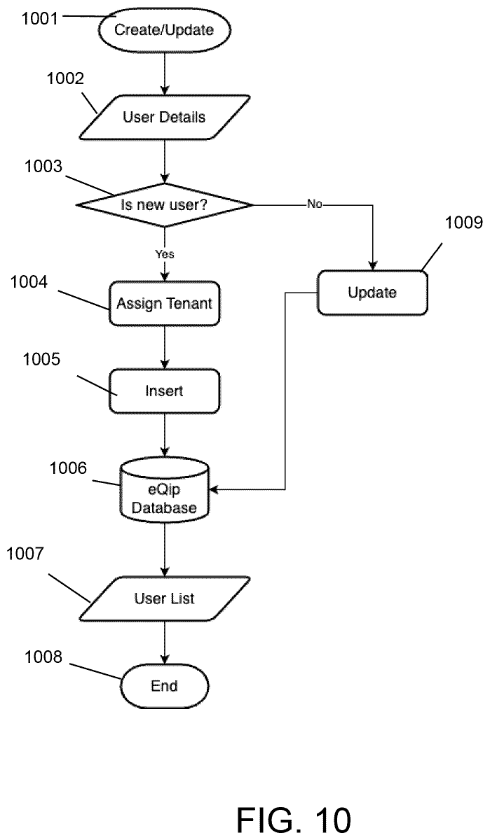

FIG. 10 is a flow chart showing an exemplary process for a user accessing the software for electronic quality inspection as an individual, according to an aspect. As an example, a user can assign tenants in the software. As used herein, “tenant” may refer to firms, companies, organizations, and the like. First, the user can create or update a user profile (step 1001). The software will then retrieve user details (step 1002), and check if this is a new user (step 1003). If yes, the software will assign a tenant to the user (step 1004), which signifies a user as working for or otherwise being associated with the tenant. Next, the information on the new tenant can be inserted into the system (step 1005) and stored in the eQip database (step 1006). A user list can be generated from the AWS cognito 703 and eQip databases 706 (step 1007), and then the process can then be completed (step 1008). If the user is not determined to be a new user in step 1003, the system can then prompt for whether user details should be updated (step 1009), and proceed to store the information in the database (step 1006).

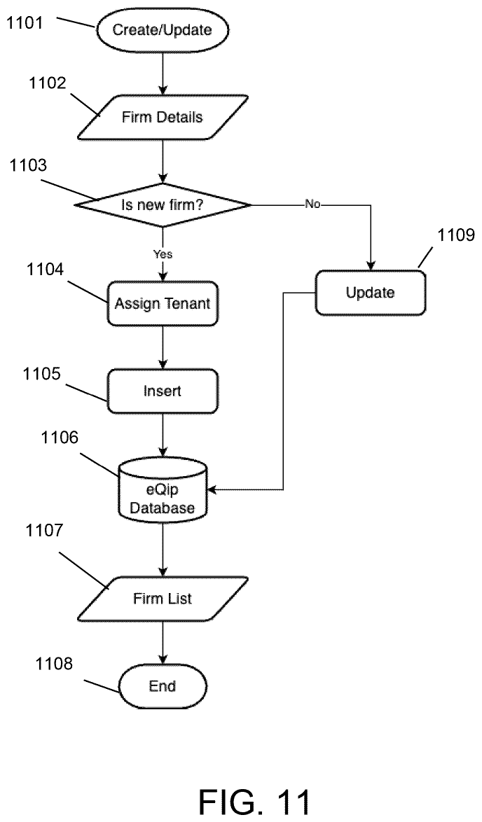

FIG. 11 is a flow chart showing an exemplary process for a user accessing the software for electronic quality inspection on behalf of or acting as a firm, according to an aspect. First, the user can create or update a firm profile (step 1101). The software will then retrieve user details (step 1102), and check if this is a new firm (step 1103). If yes, the software will assign a tenant to the firm (step 1104). Next, the information on the new tenant can be inserted into the system (step 1105) and stored in the eQip database (step 1106). A firm list can be generated from the database (step 1107), and then process can then be completed (step 1108). If the firm is not determined to be a new firm in step 1103, the system can then prompt for whether firm details should be updated (step 1109), and proceed to store the information in the database (step 1106).

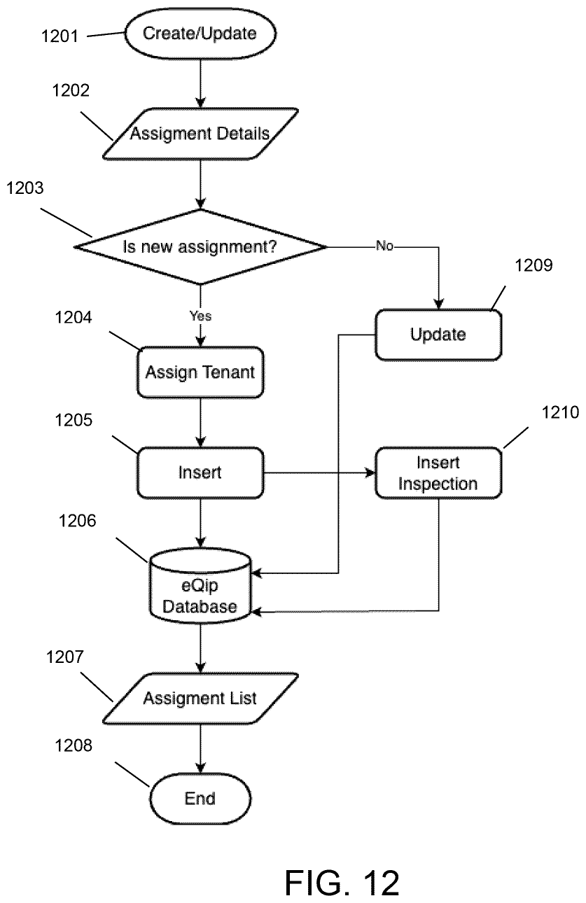

FIG. 12 is a flow chart showing an exemplary process for creation of an assignment by using the software for electronic quality inspection, according to an aspect. First, the user can create or update an assignment (step 1201). The software will then retrieve assignment details (step 1202), and check if this is a new assignment (step 1203). If yes, the software will assign a tenant to the assignment (step 1204) ensuring that an assignment is tied to a particular tenant. Next, the information on the new tenant can be inserted into the system (step 1205) and stored in the eQip database (step 1206). An assignment list can be generated from the database (step 1207), and then process can then be completed (step 1208). If the assignment is not determined to be a new assignment in step 1203, the system can then prompt for whether assignment details should be updated (step 1209), and proceed to store the information in the database (step 1206). Additionally, new inspections can be performed and details from a new inspection can be inserted (step 1210) to be added to existing assignments. This information can then be added to the eQip database (step 1206).

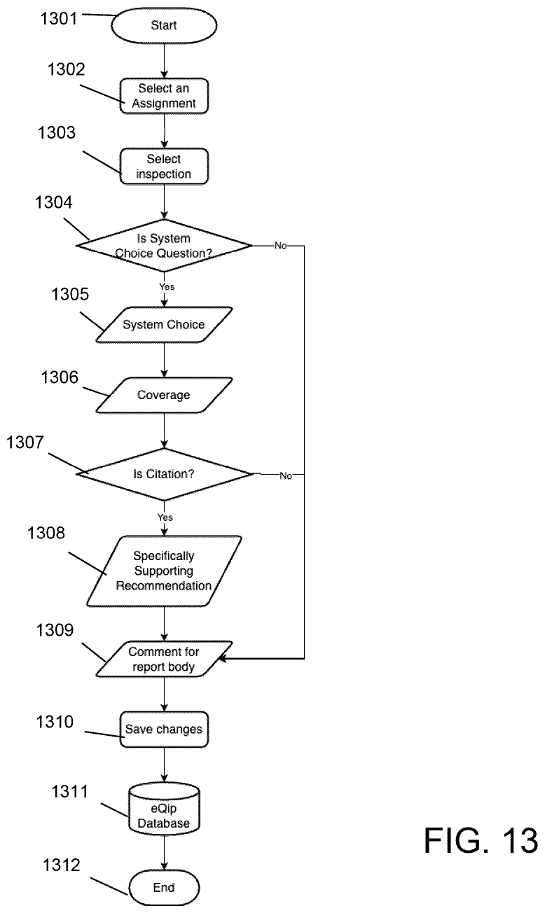

FIG. 13 is a flow chart showing an exemplary process for performing an inspection by using the software for electronic quality inspection, according to an aspect. First, the user can begin by accessing the eQip portal through any suitable means (step 1301). Next, the user may select an assignment (step 1302), and from there, select an inspection (step 1303). Next, the system will check for whether there is a “choice question,” which refers to providing the user with a choice as to which system the observation or findings belong to. If “yes,” the user can then make a selection (step 1305) and begin coverage of the assignment (step 1306). The system can then prompt the user to check whether a citation should be included (step 1307). If “yes,” the user can then be prompted to provide specific supporting information and recommendations relating to the citation (step 1308). The user can next be prompted to add a comment for the report body (step 1309). Next, the user can save changes (step 1310). The transaction, which refers to all information entered and saved relating to the above steps, can then be stored in the eQip database (shown as 710 in FIG. 7; step 1311). Then, the process can be ended (step 1312). If “no” in step 1304, the user can proceed to step 1309 and continue the process as described above. If “no” in step 1307, the user can proceed to step 1309 and continue the process as described above.

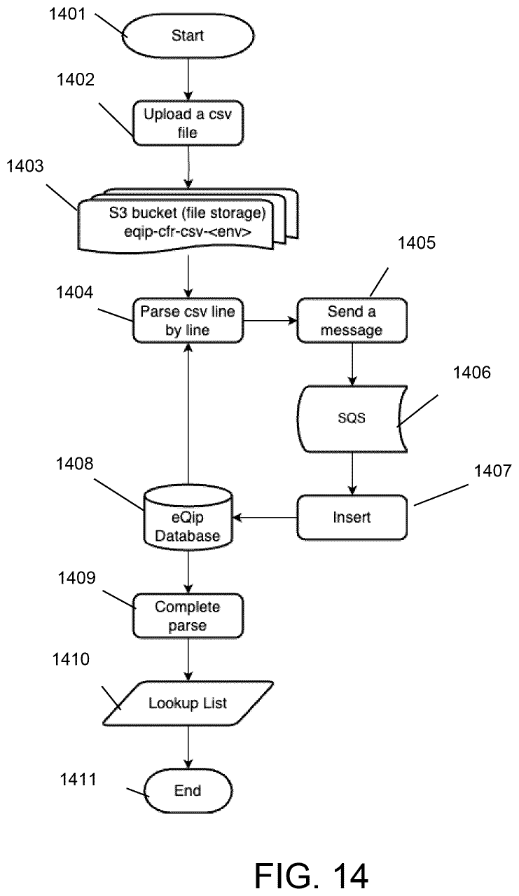

FIG. 14 is a flow chart showing an exemplary process for performing a lookup using the software for electronic quality inspection, according to an aspect. Generally, this process provides a method for uploading a file such as a spreadsheet in which regulations are embedded. As discussed above, spreadsheets can be uploaded to the system and become stored in the file storage system (shown as 709 in FIG. 7) of the software system. First, the process can be started (step 1401) by accessing the software for electronic inspection. Next, a user can upload spreadsheet, which can, for example, be in csv format (step 1402). Next, this file can be stored in the S3 bucket (709 in FIG. 7; step 1403). Next, the system may be configured to parse the csv line by line (step 1404) and send a message to the queue system (step 1405). The queuing may be managed by the SQS (717 in FIG. 7; step 1406), which can then insert the requested actions (step 1407) and then send the information from the csv into the eQip database (step 1408). The parse can then be completed by the system (step 1409), and a lookup list can be created from the uploaded csv (step 1410), and the process can then be ended (step 1411).

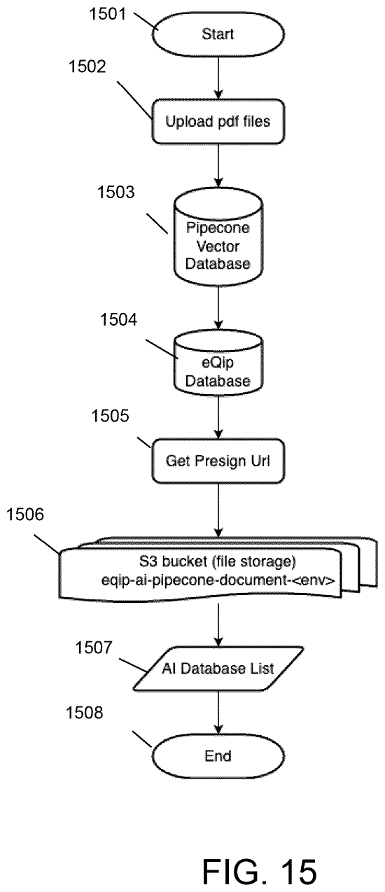

FIG. 15 is a flow chart showing an exemplary process for adding information to a database of the software for electronic quality inspection, according to an aspect. First, the user can begin by accessing the eQip portal through any suitable means (step 1501). Next, the user can directly upload PDF files (step 1502) which, as described above when referring to FIG. 7, can be stored in the S3 file storage system. The system may be configured to automatically convert the information contained in the PDF files into binary for storage in the Pinecone vector-based database (step 1503), which can also be in communication with the Postgres database (step 1504). eQip database. Next, a presign URL is obtained (step 1505). A presign URL can be used for temporary access to a particular PDF file. As an example, a user may input a question to the system and be provided with an AI-generated answer, which can also provide references (such as, for example, the scenario shown in FIG. 2B). The system may be configured to provide a presign URL, which can expire after a predetermined length of time, that a user can click on to be redirected to a PDF file, for example, which generally may be stored in the S3 file storage system (step 1506), and which can be associated with a AI database list (step 1507). The process can then be ended (step 1508).

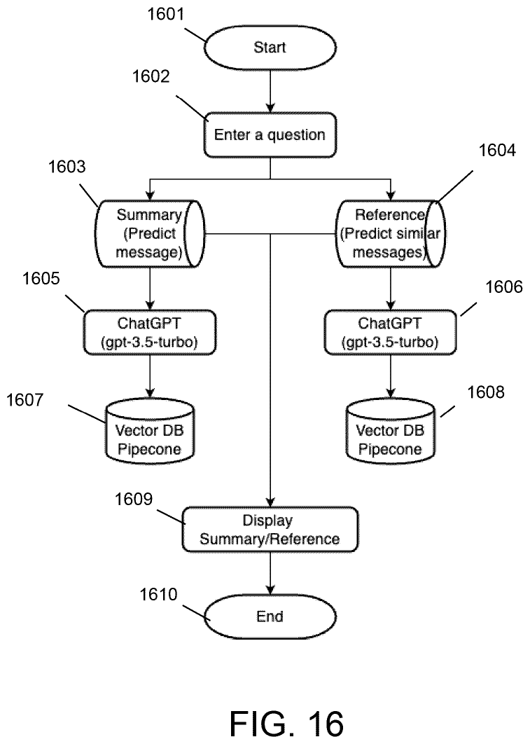

FIG. 16 is a flow chart showing an exemplary process for asking questions and receiving answers from the AI database of the software for electronic quality inspection, according to an aspect. First, a user can access the eQip web portal through any suitable means (step 1601). Next, the user can enter a question (step 1602). The software can then provide an answer summary (step 1603) and provide references (step 1604). The summary can be provided from generative text AI (step 1605) and the references can be provided from generative text AI (step 1606). These can each be derived from data stored on the vector based database (steps 1607 and 1608). Next, these summary and reference data can be displayed to the user (step 1609) as discussed above when referring to FIG. 15, and then the process can be closed and ended (step 1610).

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

Claim limitations should be construed as means-plus-function limitations only if the claim recites the term “means” in association with a recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

What is claimed is:

1. A system for assessing adherence to pharmaceutical regulations and performing inspections of pharmaceutical facilities, the system comprising:
a database server that includes a file storage system configured to store data received from data entry, a first database configured to store data retrieved from the file storage system in a vector-based format, and a second database configured to store data related to transactions performed using the system;
a software module having a connection to the database server and to an online network;
a user interface accessible through a computing device, the user interface enabling communication with the database server;
the system being configured to perform operations comprising:
performing the data entry by receiving information from a plurality of sources, wherein the plurality of sources comprise data relating to regulations provided by a plurality of official regulatory bodies uploaded into the file storage system by a first preauthorized user;
converting the data received from the data entry into vector-based format and storing the converted data in the first database;
receiving an input query and generating an output answer in response to the input query by using the data stored in the first database;
providing reference materials relating to the output answer, wherein the reference materials are pulled from the data received from the data entry;
providing guided tutorials for a plurality of tasks, wherein at least a portion of the guided tutorials are generated from the data received from the data entry;
providing access to the data received from the data entry according to a second preauthorized user's access level;
creating assignments associated with an entity;
controlling access by a plurality of users to the assignments of the created assignments, such that each user of the plurality of users is provided with a tier level of access, wherein the system is configured to allow access by multiple users to an individual assignment, such that simultaneous access to the individual assignment by more than one user is possible; and
creating an inspection report related to the individual assignment by compiling assignment information provided by at least one user having access to the assignment.

2. The system of claim 1, wherein the information from a plurality of sources comprise publicly available data relating to pharmaceutical manufacture and use.

3. The system of claim 1, wherein each of the official regulatory bodies is associated with a governing body of a country.

4. The system of claim 1, wherein an individual assignment of the created assignments requests adherence to regulations provided by at least one official regulatory body of the plurality of official regulatory bodies.

5. The system of claim 1, wherein an individual assignment of the created assignments requests adherence to regulations provided by at least two official regulatory bodies of the plurality of official regulatory bodies.

6. The system of claim 1, wherein the inspections of pharmaceutical facilities comprise audits of the entity; inspections of equipment, inspections of operating protocols and practices within the pharmaceutical facilities, and inspection of manufacturing processes within the pharmaceutical facilities.

7. The system of claim 1, wherein the entity is a company.

8. The system of claim 1, wherein the plurality of tasks comprises assessments of adherence to regulations provided by a selected regulatory body, walk-through inspections of a pharmaceutical facility, and completion of citation reports.

9. A method for assessing adherence to pharmaceutical regulations and performing inspections of pharmaceutical facilities, operable on a system including a computing device comprising a database server, a processor, a display, and a camera, the database server having: a file storage system configured to store data received from data entry, a first database configured to store data retrieved from the file storage system in a vector-based format, and a second database configured to store data related to transactions performed using the system; the method comprising:
performing data entry by receiving information from a plurality of sources, wherein the plurality of sources comprise data relating to regulations provided by a plurality of official regulatory bodies uploaded into the file storage system by a first preauthorized user;
converting the data received from the data entry into vector-based format;
storing the converted data in the first database;
providing a user interface accessible through the computing device, wherein the user interface enables communication with the database server;
creating assignments associated with an entity;
controlling access by a plurality of users to the assignments of the created assignments, such that each user of the plurality of users is provided with a tier level of access, wherein the system is configured to allow access by multiple users to an individual assignment, such that simultaneous access to the individual assignment by more than one user is possible;
receiving assignment information from the plurality of users;
compiling the assignment information; and
creating an inspection report related to the individual assignment from the assignment information provided by at least one user of the plurality of users having access to the assignment.

10. The method of claim 9, further comprising receiving visual information and audio information from the camera, recording the visual information and the audio information, and storing the recorded visual information and the recorded audio information in the file storage system.

11. The method of claim 9, comprising:
connecting a first user to the system via a first computing device;
connecting a second user to the system via a second computing device;
allowing communication between the first user and the second user through the user interface accessed on the first computing device and the user interface accessed on the second computer device.

12. The method of claim 9, wherein an individual assignment of the created assignments requests adherence to the regulations provided by at least one official regulatory body of the plurality of official regulatory bodies.

13. The method of claim 9, wherein an individual assignment of the created assignments requests adherence to the regulations provided by at least two official regulatory bodies of the plurality of official regulatory bodies.

14. The method of claim 9, wherein the plurality of sources comprises publicly available data relating to pharmaceutical manufacture and use.

15. The method of claim 9, further comprising receiving an input query, and generating an output answer in response to the input query by using the data stored in the first database.

16. A method for collaboratively assessing adherence to pharmaceutical regulations and performing inspections of a pharmaceutical facility, operable on a system including a computing device comprising a database server, a processor, a display, and a camera, the database server having: a file storage system configured to store data received from data entry, a first database configured to store data retrieved from the file storage system in a vector-based format, and a second database configured to store data related to transactions performed using the system; the method comprising:
performing data entry by receiving information from a plurality of sources, wherein the plurality of sources comprise data relating to regulations provided by a plurality of official regulatory bodies uploaded into the file storage system by a first preauthorized user;
converting the data received from the data entry into vector-based format;
storing the converted data in the first database;
providing a first user interface accessible through a first computing device, and providing a second user interface accessible through a second computing device, wherein the first user interface and the second user interface each enable communication with the database server and enable communication with each other;
creating an assignment associated with an entity related with the pharmaceutical facility;
controlling access by at least the first user and the second user to the assignment, such that simultaneous access to the assignment by the first user and the second user is possible;
receiving visual information and audio information from the camera of the first computing device and communicating the visual information and the audio information to the second computing device;
recording the visual information and the audio information;
storing the recorded visual information the recorded audio information in the file storage system;
receiving assignment information from the first user and the second user;
compiling the assignment information; and
creating an inspection report related to the assignment from the assignment information provided by the first user and the second user.

17. The method of claim 16, wherein the second user is remote to the pharmaceutical facility.

18. The method of claim 16, wherein the assignment requests adherence to the regulations provided by at least one official regulatory body of the plurality of official regulatory bodies.

19. The method of claim 16, wherein the assignment requests adherence to the regulations provided by at least two official regulatory bodies of the plurality of official regulatory bodies.

20. The method of claim 16, wherein the inspections of pharmaceutical facility comprise audits of the entity; inspections of equipment, inspections of operating protocols and practices within the pharmaceutical facility, and inspection of manufacturing processes within the pharmaceutical facility.
